(19) United States
(12) Patent Application Publication
(10) Pub. No.: US 2004/0253300 A1
(43) Pub. Date: Dec. 16, 2004

(54) Medication and method for remediating existing scars through transdermal, topical delivery of calcium channel blockers

(76) Inventor: W. Jerry Easterling, San Antonio, TX (US)

Correspondence Address: David G Henry, 900 Washington Avenue, PO Box 1470, Waco, TX, 76703-1470, US

(21) Appl. No.: 10/493,784

(22) PCT Filed: Oct. 24, 2002
(86) PCT No.: PCT/US02/34063

Related U.S. Application Data
(63) Continuation-in-part of application No. 10/044,783, filed on Oct. 24, 2001, now abandoned.
Application No. 10/044,783 is a continuation-in-part of application No. 09/411,175, filed on Oct. 1, 1999, now Pat. No. 6,353,028.
Application No. 09/411,175 is a continuation-in-part of application No. 09/128,103, filed on Aug. 3, 1998, now Pat. No. 6,031,005.

(51) Int. Cl. A61K 31/554; A61K 9/70

(52) U.S. Cl. 424/449; 514/211.07; 514/355

(57) ABSTRACT

The invention is of a non-invasive, topical medicament and associated methodology for use thereof, through the use of which existing scars may be effectively, cost effectively, and painlessly treated. One or more calcium channel blocker agents serve as the primary active ingredient of the present compositions and transdermal penetration agents or carriers are included to facilitate topical delivery of the active ingredient(s) to the intended, sub-dermal treatment site.

### CITATION TO PRIOR APPLICATION

This is a continuation application with respect to U.S. application, Ser. No. 10/044,783, filed 24 Oct. 2001 from which priority is claimed under 35 U.S.C. §120 and under provisions of the Patent Cooperation Treaty.

### BACKGROUND OF THE INVENTION

1. Field of the Invention

Applicant's invention relates to medicaments and treatment procedures relating to scars arising from trauma, surgery, and burns.

2. Background Information

In U.S. Pat. No. 6,031,005 (and subsequently filed continuation-in-part applications in relation thereto, which CIPs have not issued at the time of this filing), the present inventor has provided new and unobvious treatment regimens for a variety of fibrotic conditions through the use of topically applied calcium channel blocker-based preparations. The specification of U.S. Pat. No. 6,031,005 (“the '005 patent”) is incorporated herein by reference, as if set forth herein verbatim.

The preparations and associated methods for the topical application of calcium channel blocker preparations as taught in the '005 patent have proven remarkably effective in treating, not only the conditions specified in the claims, but, as indicated therein (albeit not in the detail set forth herein), in remediating existing scars.

Scarring represents a major challenge to medical science. Scars are almost universally abhorred, and countless billions of dollars are spent in minimizing or reversing their appearance.

Certain work has been done, even with respect to the use of calcium channel blockers, in preventing scarring. However, the prior attempts have failed to take into consideration factors which, as discovered by the present inventors, greatly enhance the ability to remediate scars, regardless of their age.

Examples of prior approaches to preventing scarring which, if followed, would not achieve the claimed result, are shown in the Green patents (U.S. Pat. Nos. 5,902,609 and 5,569,678).

Significant distinctions between the present invention and those of the cited Lee patents include: (1) that Lee teaches injection of calcium channel blockers (as opposed to topical application; and (2) Lee fails to recognize and put before the public the significance of using calcium channel blockers at a time when the inflammatory stage of wound healing has passed (in other words, when scar tissue has already formed).

With respect to the first point: the present inventor's experience in topical treatment of such conditions as Peyronie's Disease and other fibrotic conditions, has taught him that topical, transdermal delivery of calcium channel blocker agents to treat aberrant fibrotic tissue accumulations is far more efficacious than invasive applications or deliveries. Most scar tissues are quite dense, and injection of calcium channel blockers direction into the fibrotic tissue tends not to be effective in remodeling the tissue. Not only is dissemination of the effective agent impeded by the comparative density of the tissue, but the calcium channel blocker-induced processes which result in “tissue remodeling” for remediation of scar tissue are not effectively initiated from within the tissue itself. Rather, for reasons not yet fully understood, approaching the targeted tissue from its perimeter promotes the desired of biological activities to a remarkably greater degree. The most effective way thus found to achieve this peripheral attack on scar tissue is to deliver the calcium channel blockers transdermally. This, in turn, requires that the calcium channel blocker medication be formulated for transdermal delivery.

Neither the benefits of peripheral attack of scar tissue, nor the associated benefits of transdermal delivery of calcium antagonists are revealed or suggested in any known item of prior art.

With respect to the second point: the application of calcium channel blocker preparations to actual wounds will inhibit the healing process. This is counter- productive in any wound management approach. Nothing in the prior art suggests that anyone has possessed or suggested that one should wait until scar tissue has already formed to use calcium antagonists to remediate the scar.

The present inventor has learned that the topically, transdermally delivered calcium channel blocker-based medications of his invention are quite efficacious in remediating even old, existing scars, but almost as significantly, that such is (contrary to the inferred teachings of Lee) contraindicated prior to approximately two weeks from a would-producing event (surgery, contusion trauma, etc.).

The preferred embodiment and mode of the present invention involves, not just any calcium channel blocker-based medication which is combined with suitable transdermal delivery agents, but is formulated for stability at the calcium antagonist dosage ranges which are found most efficacious for treating (existing) scars.

The heretofore preferred embodiment and best known mode of the topical calcium channel blocker preparations as taught in the '005 patent, both in 10% and 15% strengths, have demonstrated instability as evidenced by the formation of crystals, at which point the medication becomes significantly less efficacious and, in the case of use on “young” scars, may impart needless pain from abrasion on application.

The time over which crystals have formed in topical verapamil formulations has varied dramatically, from as little as 30 days to as long as 90 days after others do not appear to have done so. These inconsistencies suggested to the present inventor that the chemical reactions involved were initiated by more than one material cause.

Prior to deterioration, the topical calcium antagonists preparations as taught by the '005 patent performed beyond anyone's reasonable expectations in treating various aberrant fibrotic conditions. However, once deterioration reaches a detectable level, difficulty in accurately dispensing the preparations comes into play and at least calls into question the level of efficacy to be expected from use of the preparations because of the apparent chemical changes having occurred (with possible reduction in active ingredients).

Therefore, it became imperative, at least in the view of the present inventor, that a new formulation be found which would alleviate the deterioration problem with the topical calcium antagonist formulations, particularly if the formulations were to be distributed as an FDA-approved, off-the-shelf pharmaceutical product, rather than a formulated-upon-demand (by prescription) medication. Of course, such new formulation should not be made at the expense of efficacy.

Calcium antagonist preparations made as prescribed herein have proven uniquely effective in treating a number of fibrotic conditions or manifestations, including the primary topic of this application—existing scarring.

### SUMMARY OF THE INVENTION

It is an object of the present invention to provide an medicament useful in the treatment of existing scar tissue.

It is another object of the present invention to provide an topical medicament for the treatment of existing scars.

It is another object of the present invention to provide an topical medicament for the treatment of existing scars, which topical medicament effects tissue remodeling through the non-invasive, transdermal delivery of calcium antagonist agents to scar tissue.

It is another object of the present invention to provide an topical medicament for the treatment of existing scars, which topical medicament effects tissue remodeling through the non-invasive, transdermal delivery of calcium antagonist agents to scar tissue and is formulated to provide long-term stability at optimal calcium antagonist dosage levels.

It is another object of the present invention to provide a method for remediation of existing scars through non-invasive, transdermal delivery of calcium antagonist agents to scar tissue.

In satisfaction of these and related objectives, Applicant's present invention provides an topical medicament and associated methodologies for preparation and use thereof, through the use of which medicament, via topical application and resulting transdermal delivery of calcium channel blocker agents to scar tissue, scar tissue is remediated or “remodeled” to reduce the visual and palpable presence of scars. The present medicaments and associated methods are applicable to both new and long-existing scars.

The invention, although exemplified by specific embodiments which are based upon, or rely on the use of specific calcium channel blockers, is not limited to such species. Rather, observations by the present inventor indicate that when coupled with a suitable carrier for transdermal delivery, all thus-far-evaluated calcium channel blocker-based preparations (regardless of the species of calcium channel blocker used) effect the desired remodeling of existing scar tissue. Therefore, the true scope of the invention encompasses preparations and methods of use in facilitating, or involving the use of topical, transdermal application of calcium channel blockers in the treatment of existing scars.

The medicament of the present invention is an quite shelf-stable topical gel which, like its predecessor as taught in the '005 patent, repeatably and predictably effects substantial remediation of scars to achieve an aesthetically positive change in scar appearance.

### DETAILED DESCRIPTION OF THE PREFERRED EMBODIMENT

In the preferred embodiment of the present medicament, and in the medicament upon which the associated method are based, the primary active ingredient is Verapamil Hydrochloride, USP (a diphenylalkylamine). However, it should be understood that other calcium channel blockers (topically applied in a similar composition) provide similar results. With certain patients, combinations of channel blocker agents seem to have an even greater efficacy than the single, Verapamil agent. Other such calcium channel blockers include benzothiazepines (Diltiazem, for example), dihydropyridines (Amlodipine, Felodipine, Isradipine, Nicardipine, Nifedipine, Nimodipine, or Nisoldipine), and the fast sodium inward channel inhibitor—Bepridil. Diltiazem in particular, has proven effective when substituted for Verapamil, particularly for patients with a demonstrated skin sensitivity to Verapamil. Appropriate dosage substitutions when substituting one particular calcium antagonist for another (Verapamil for Diltiazem, for example) will be made in same manner as if such agents were being interchanged for their existing, more conventional uses). Likewise, combining multiple calcium antagonists will result in similar dosage considerations, as will be apparent to persons skilled in the art.

I. Basis of Formulation Changes

In evaluating the deterioration problems with the prior embodiments of the present inventor's medicaments, the present inventor may get the following observations and/or came to certain conclusions:

1. Air is being entrained into the materials at all stages of formulation.

The ethoxydiglycol reagent is reacting with the air and forming byproducts including but not limited to aldehydes, peroxides, and free radicals which cause drug crystallization and subsequent loss of therapeutic potency. Additionally, these byproducts can cause skin irritation.

Verapamil is a chemical derivative of papaverine. Papaverine, in the presence of heavy metals, will deteriorate rapidly. The verapamil formulations may be affected by the presence of heavy metal ions that originate from the mixing containers or equipment.

Based upon these conclusions, the present inventor made the following basic changes to his prior formulations and preparation steps:

1. Butylated hydroxytoluene (BHT), NF. BHT is added, and serves as an antioxidant to counteract any reaction with entrained air.

2. Nitrogen, NF, is used to purge all containers during chemical addition and mixing. Every ointment tube is purged just prior to filling and sealing. The nitrogen serves as a replacement for entrained air and is non-reactive with the components.

3. A “non-reactive” glaminate ointment tube is used so that no reaction occurs with the ointment tube.

4. Edetate disodium, USP is added to the gel formulation and serves as a chelating agent to bind any heavy metal ions and prevent reaction of same.

5. Propylene glycol, USP has been added as an additional drug solvent and skin absorption enhancer.

The result of maling the preceding changes to the prior gel formulations is a gel which is stable over periods of many months, even after undergoing formal, rigorous stability studies by an independent pharmaceutical laboratory. Patient evaluations indicate that the change in formulation has in no way negatively affected efficacy and, and fact, appears to have somewhat enhanced such efficacy.

II. Preparation

The now-preferred Verapamil-based gels of the present invention (in exemplary 10% and 15% percent strengths) may be prepared according to the following disclosure and protocol, with variations appropriate to a desired scale of production as will be apparent to persons skilled in the production of pharmaceutical preparations:

A. Constituents of Preferred Embodiment of Topical Verapamil
Gel 10% and 15%

| Ingredients | 10% (% W/W) | 15% (% W/W) |
|---|---|---|
| Verapamil | 10.0 | 15.0 |
| Ethoxydiglycol | 14.0 | 19.5 |
| Propylene Glycol | 0.5 | 0.5 |
| Butylated Hydroxy Toluene (BHT) | 0.1 | 0.1 |
| Lecithin Soya Granular | 13.1 | 13.1 |
| Isopropyl Myristate | 13.1 | 13.1 |
| Sorbic Acid | 0.09 | 0.09 |
| Pluronic F127 | 9.8 | 11.6 |
| Potassium Sorbate | 0.15 | 0.12 |
| Disodium Edetate | 0.01 | 0.01 |
| Purified Water | 39.15 | 26.88 |

B. Topical Verapamil 15% (To Make 3000 Gm).

| Ingredients | Quantity | |
|---|---|---|
| Verapamil HCI USP | 450.00 | Gm |
| Ethoxydiglycol Reagent | 585.0 | Gm |
| Lecithin/lsopropyl Myristate Solution | 790.0 | Gm |
| Butylated Hydroxytolune NF (BHT) | 3.0 | Gm |
| Edetate Disodium USP | 0.30 | Gm |
| Propylene Glycol USP | 15.0 | Gm |
| Pluronic Gel 30% | 1,156.7 | Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 10 minutes using a 3 inch mixing blade at 3100 rpm. Dispense in 30 Gm glaminate ointment tubes.

C. Topical Verapamil 10% (To Make 3000 Gm).

| Ingredients | Quantity | |
|---|---|---|
| Verapamil HCI USP | 300.00 | Gm |
| Ethoxydiglycol Reagent | 420.0 | Gm |
| Lecithin/lsopropyl Myristate Solution | 790.0 | Gm |
| Butylated Hydroxytolune NF (BHT) | 3.0 | Gm |
| Edetate Disodium USP | 0.30 | Gm |
| Propylene Glycol USP | 15.0 | Gm |
| Pluronic Gel 30% | 1,471.7 | Gm |

Instructions: Dissolve verapamil in ethoxydiglycol and propylene glycol with the aid of heat (90-100 degrees C). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 5 minutes using a 3 inch mixing blade at 3100rpm. Dispense in 30Gm glaminate ointment tubes.

D. Pluronic Gel 20% (To Make 3000 Gm)

| Ingredients | Quantity | |
|---|---|---|
| Pluronic F127 NE (Poloxamer 407) | 600.00 | Gm |
| Potassium Sorbate NE | 9.00 | Gm |
| Water (Sterile for Irrigation) qs to | 3,000.00 | Gm |

Directions: Prepare a pluronic gel by combining the potassium sorbate and pluronic F 127 and bringing to a total weight of 3,000 Gm. with cold (refrigerated) sterile water. Make sure that all the granules are wet, and place in a refrigerator. Mixture will form a clear solution over 2448 hours.

Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

E. Pluronic Gel 30% (To Make 2000 Gm).

| Ingredients | Quantity | |
|---|---|---|
| Pluronic F 127 NF (Poloxamer 407) | 600.00 | Gm |
| Potassium Sorbate NF | 6.00 | Gm |
| Water (Sterile for Irrigation) qs to | 2,000.00 | Gm |

Instructions: Prepare a pluronic gel by combining the potassium sorbate and pluronic F 1 27 and bringing to a total weight of 2,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

F. Lecithin/lsopropyl Myristate Solution (To Make 3000 Gm).

| Ingredients | Quantity | |
|---|---|---|
| Lecithin Soya Granular | 1,494.0 | Gm |
| Isopropyl Myristate NF | 1,494.0 | Gm |
| Sorbic Acid NF Powder | 9.90 | Gm |

Instructions: Disperse lecithin and sorbic acid in isopropyl myristate. Allow to stand at room temperature until a liquid of syrup consistency forms. Stir well and store in a light protected container.

III. Use of Preparations

The choice of strengths of the topical calcium antagonist gels taught above will depend on the experience of the clinician. Ordinarily, a patent with a to-be-treated scar will be started with the lower dosage preparation, and only if the patient fails to respond, or responds more slowing than reasonably would be expected, would the patient be changed to the higher dosage form.

In any event, use of all topical calcium channel blocker preparations of the present inventor's work involves simply applying a thin coating of the gels to, and immediately surrounding a scar. For a particularly large scar area, a practitioner may want to treat first one portion, then subsequent other portions of a scar, if excessive, systemic absorption of the calcium antagonist agent is for some reason contraindicated and complete coverage of a patient's scar(s) may expose the patient to a greater than desired gross daily dosage of the calcium antagonist(s).

Particularly in cases of possible contraindications to greater than certain levels of dosages, clinicians may prescribe certain volumetric dosages, which dosages can be metered by any number of conventional metering means (syringes, dosimeters, blister packs, single-dose tubes, etc.). Ordinarily, a once-daily application will be sufficient to achieve desired results in a matter of a few weeks to as much as six months in some cases (based on experience to date). If there are no contraindications for possible greater systemic absorption for any given patient, and faster results are desired and not otherwise contraindicated, a twice daily regimen may be used, once patient tolerance is established at the once daily level.

As referenced earlier, satisfactory results in scar remediation can only be achieved if a certain degree of healing has already occurred. Stated differently, any scar-producing event involves an inflammatory stage, during which calcium antagonists have limited, if not counter-productive effects. Certainly, in the case of wounds from surgery, traumatic lacerations, etc., exposure of the wound site to calcium antagonists is to be avoided (again, contrary to the teachings of those contributing to the prior art). Therefore, use of the present medicaments and practice of the associated methodologies should not ordinarily be done until approximately two weeks from the wound-producing event, or until scar production is observable, whichever occurs later.

Although the invention has been described with reference to specific embodiments, particularly with respect to the particular active ingredient of the present medicament, this description is not meant to be construed in a limited sense, in particular to limit the scope of the appended claims to cover only those medicaments and associated modalities of treatment which include Verapamil as the calcium channel blocker, the function of which in the area of plaque appears to lie at the heart of the efficacy of the present medicament. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A topical, transdermal medicament for use in treating existing scars comprising:
a carrier host agent for facilitating non-invasive, transdermal delivery of a calcium antagonist into scar tissue;
a therapeutic dosage of a calcium channel blocker agent suspended in said carrier host agent.

2. The medicament of claim 1 further comprising:
an antioxidant agent suspended in said carrier host agent for preventing the oxidation of active ingredients of said medicament.

3. The medicament of claim 1 wherein said calcium channel blocker agent is verapamil.

4. The medicament of claim 1 wherein said calcium channel blocker agent is a benzothiazepine.

5. The medicament of claim 1 wherein said calcium channel blocker agent is a dihydropyridines.

6. The medicament of claim 1 wherein said calcium channel blocker agent is Nifedipine.

7. The medicament of claim 1 wherein said medicament comprises:
verapamil;
a lecithin/isopropyl myristate solution;
butylated hydroxy toluene;
pluronic F127; and
water.

8. The medicament of claim 1 wherein said medicament comprises:
one or more calcium antagonist selected from the calcium channel blocker categories of diphenylalkylamines, benzothiazepines, and dihydropyridines;
a lecithin/isopropyl myristate solution;
butylated hydroxy toluene;
pluronic F127; and
water.

9. The medicament of claim 1 wherein said medicament comprises:
a diphenylalkylamine calcium antagonist agent;
a lecithin/isopropyl myristate solution;
butylated hydroxy toluene;
pluronic F127; and
water.

10. The medicament of claim 7 further comprising:
Edetate disodium.

11. The medicament of claim 8 further comprising:
Edetate disodium.

12. The medicament of claim 9 further comprising:
Edetate disodium.

13. The medicament of claim 10 further comprising:
Propylene glycol.

14. The medicament of claim 11 further comprising:
Propylene glycol.

15. The medicament of claim 12 further comprising:
Propylene glycol.

16. A method for remediating existing scars comprising the steps of:
selecting a medicament comprising:
a carrier host agent for facilitating non-invasive, transdermal delivery of a calcium antagonist into scar tissue;
a therapeutic dosage of a calcium channel blocker agent suspended in said carrier host agent;
periodically, topically applying a therapeutic dosage of said medicament to the surface of an existing scar.

17. The method of claim 16 wherein said calcium channel blocker agent is verapamil.

18. The medicament of claim 16 wherein said calcium channel blocker agent is a benzothiazepine.

19. The medicament of claim 16 wherein said calcium channel blocker agent is a dihydropyridines.

20. The medicament of claim 16 wherein said calcium channel blocker agent is Nifedipine.

21. The medicament of claim 16 wherein said medicament comprises:
verapamil;
a lecithin/isopropyl myristate solution;
butylated hydroxy toluene;
pluronic F127; and
water.

22. The medicament of claim 16 wherein said medicament comprises:
one or more calcium antagonist selected from the calcium channel blocker categories of diphenylalkylamines, benzothiazepines, and dihydropyridines;
a lecithin/isopropyl myristate solution;
butylated hydroxy toluene;
pluronic F127; and
water.

23. The medicament of claim 16 wherein said medicament comprises:
a diphenylalkylamine calcium antagonist agent;
a lecithin/isopropyl myristate solution;
butylated hydroxy toluene;
pluronic F127; and
water.

24. The medicament of claim 21 further comprising:
Edetate disodium.

25. The medicament of claim 22 further comprising:
Edetate disodium.

26. The medicament of claim 23 further comprising:
Edetate disodium.

27. The medicament of claim 24 further comprising:
Propylene glycol.

28. The medicament of claim 25 further comprising:
Propylene glycol.

29. The medicament of claim 26 further comprising:
Propylene glycol.
